(19) United States
(12) Patent Application Publication
(10) Pub. No.: US 2007/0149782 A1
(43) Pub. Date: Jun. 28, 2007

(54) Methods of preparing a crystalline form of 7-(4-chlorobutoxy)-3,4-dihydro-2(1h)-quinolinone and the use thereof in the synthesis of Aripiprazole

(76) Inventors: Michael Brand, RaAnana (IL); Moty Shookrun, Petach-Tikva (IL); Irina Gribun, Bat-Yam (IL); Itai Adin, Beer-Sheva (IL); Carmen Iustain, Beer-Sheva (IL); Oleg Braverman, Beer-Sheva (IL); Natalia Udis, Beer-Sheva (IL); Oded Arad, Rechovot (IL); Joseph Kaspi, Givatayim (IL)

Correspondence Address: Martin D. Moynihan;PRTSI, Inc., P.O. Box 16446, Arlington, VA, 22215, US

(21) Appl. No.: 11/315,230

(22) Filed: Dec. 23, 2005

(51) Int. Cl. A61K 31/496; C07D 403/02

(52) U.S. Cl. 544/363; 546/158

(57) ABSTRACT

The present invention relates to methods of preparing a highly pure crystalline form of 7-(4-chlorobutoxy)-3,4-dihydro-2(1H)-quinolinone, which is a chemical intermediate useful in the preparation of Aripiprazole thereof in high quality and yield, and provides data that characterizes the crystalline form of 7-(4-chlorobutoxy)-3,4-dihydro-(1H)-quinolinone.

### FIELD OF THE INVENTION

The present invention relates to methods of preparing a crystalline form of 7-(4-chlorobutoxy)-3,4-dihydro-2(1H)-quinolinone, which is a chemical intermediate useful in the preparation of Aripiprazole.

### BACKGROUND OF THE INVENTION

Aripiprazole (7-{4-[4-(2,3-dichlorophenyl)-1-piperazinyl]-butoxy}-3,4-dihydro-2(1H)-quinolinone) is represented by the following structural formula (I).

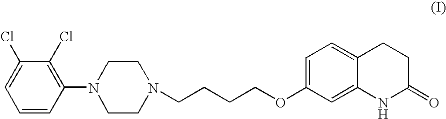

The drug is useful for treating schizophrenia and is available in tablets of different dosages.

Several synthetic methods of Aripiprazole preparation are described in U.S. Pat. No. 5,006,528 (to Otsuka Pharmaceutical Co. Ltd.), including the method illustrated in Scheme 1.

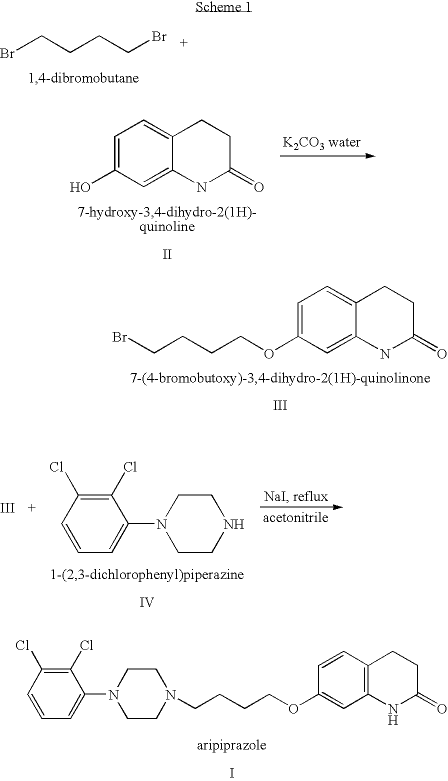

Another process for preparing Aripiprazole is described in application WO 04/063162 (to Otsuka Pharmaceutical Co. Ltd., hereinafter the ′162 application) and in U.S. patent application 2004/0192915, presented in Scheme 2 below.

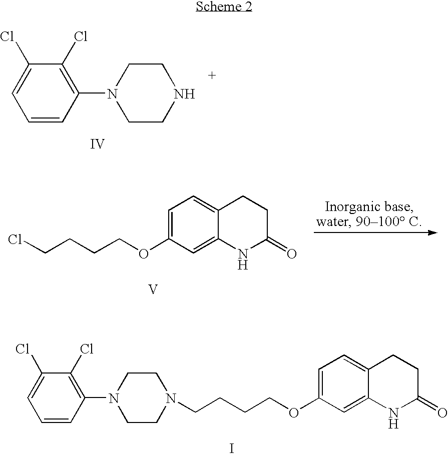

In this process, the N-alkylation of 1-(2,3-dichlorophenyl)piperazine (IV) is carried out with 7-(4-chlorobutoxy)-3,4-dihydro-(1H)-quinolinone (V, hereinafter 7-CBQ) in water in the presence of an inorganic base. A mixture of 7-CBQ, 1-(2,3-dichlorophenyl)piperazine mono hydrochloride (1.1 mole equivalents) and potassium carbonate (1.1 mole equivalents) in water (10 vol. with respect to 7-CBQ) is heated with stirring at 90-95° C. for 4 hours. Then, the reaction mixture is cooled to about 40° C., and the obtained crystals are collected by filtration. The crystals are washed with water and dissolved in ethyl acetate (9 vol.), and an azeotropic mixture of water-ethyl acetate (about 3 vol.) is distilled out. The remaining solution is cooled to 0-5° C., and the crystals are collected by filtration and dried to obtain Aripiprazole.

Although the ′162 application provides an example of Aripiprazole preparation using 7-CBQ as starting material, the preparation of 7-CBQ is not referred to in the ″162 application. Instead, the synthesis of 7-CBQ in 50% yield is mentioned by Y. Oshiro et al. in Chemical & Pharmaceutical Bulletin, 36(11), 4377-4388, (1988) and the material is characterized by its NMR spectrum and as having a melting point of 100-102° C.

In the U.S. patent application, entitled “Processes for preparing and purifying carbostyril compounds such as Aripiprazole and 7-(4-halobutoxy)-3,4-dihydro-(1H)-quinolinone”, (to Chemagis Ltd.), which claims priority from U.S. provisional patent application No. 60/617,073, filed on Oct. 12, 2004, and U.S. provisional patent application No. 60/675,444, filed on Apr. 28, 2005, which are incorporated herein by reference in their entirety as if fully set forth herein, processes are disclosed for preparing and purifying 7-(4-halobutoxy)-3,4-dihydroquinolinones (7-HBQ), which are of value as intermediates in the synthesis of Aripiprazole. Also disclosed are processes for preparing Aripiprazole, using the 7-HBQ intermediates. 7-(4-chlorobutoxy)-3,4-dihydro-(1H)-quinolinone (7-CBQ) is one such intermediate, useful in the synthesis of Aripiprazole, which when reacted with 1-(2,3-dichlorophenyl)piperazine affords Aripiprazole in high quality and yield, as described therein.

The present invention provides methods of obtaining a crystalline form of 7-(4-chlorobutoxy)-3,4-dihydro-(1H)-quinolinone, which can facilitate the production of Aripiprazole in high quality and yield.

### SUMMARY OF THE INVENTION

According to the teachings of the present invention, there are provided methods of preparing the crystalline form of 7-CBQ, by crystallizing the crude 7-CBQ, which is obtained essentially as described herein or by any other method known in the art, from different solvents or from solvent mixtures.

Thus, a preferred embodiment of the present invention relates to a method of preparing the crystalline form of 7-(4-chlorobutoxy)-3,4-dihydro-(1H)-quinolinone (7-CBQ), the method comprising:

suspending 7-CBQ in an organic solvent;

heating the suspension to elevated temperature, preferably to reflux;

allowing the thus formed solution to cool gradually;

collecting the obtained crystals by filtration; and

washing the crystals and drying, optionally under reduced pressure.

Another preferred embodiment of the present invention relates to a different method of preparing the crystalline form of 7-(4-chlorobutoxy)-3,4-dihydro-(1H)-quinolinone (7-CBQ), the method comprising:

suspending 7-CBQ in a first solvent;

heating the suspension to elevated temperature, preferably to reflux;

adding to the thus formed solution a second solvent optionally dropwise and allowing the solution to cool gradually;

collecting the obtained crystals by filtration; and

washing the crystals and drying, optionally under reduced pressure.

Another embodiment of the present invention relates to a process for purifying 7-(4-chlorobutoxy)-3,4-dihydro-(1H)-quinolinone (7-CBQ), the process comprising crystallizing the 7-CBQ, which is obtained essentially as described herein or by any other method known in the art, from a solvent or a mixture of solvents for obtaining the purified 7-CBQ, having a purity of at least about 98%, preferably a purity equal to or greater than 99%.

The present invention provides the crystalline form of 7-(4-chlorobutoxy)-3,4-dihydro-(1H)-quinolinone (7-CBQ), and a process for using this crystalline form in the preparation of Aripiprazole thereof.

One embodiment of the present invention relates to the crystalline solid comprising 7-(4-chlorobutoxy)-3,4-dihydro-(1H)-quinolinone (7-CBQ), which is characterized by unique powder X-ray diffraction pattern (Table 1, FIG. 1). The strong diffraction peaks at 8.04, 8.61, 15.24, 17.78, 19.44, 22.14, 23.27, 25.33, 25.91, and 27.18±0.2 degrees 2θ are most characteristic of this form.

The crystalline solid comprising 7-CBQ is further characterized by having a unique infra-red spectrum with characterizing absorption bands at 3195.63, 3095.34, 1675.92, 1631.56, 1594.92, 1525.49, 1461.85, 1394.95, 1380.85, 1272.85, 1199.57, 1178.35, 1062.64, 860.14, 788.78, 698.14, and 619.07±4 cm⁻¹, as depicted in FIG. 2.

The crystalline solid comprising 7-CBQ is further characterized by having a melting point of 104-105° C.

The crystalline solid comprising 7-CBQ is further characterized by having a differential scanning calorimetric (DSC) curve as depicted in FIG. 3. According to this DSC curve, the endothermic peak is at a temperature of 103.41° C., corresponding to the melting of 7-CBQ.

The crystalline solid comprising 7-CBQ is further characterized by having a TGA curve as depicted in FIG. 4.

### BRIEF DESCRIPTION OF THE FIGURES

FIG. 1 depicts the powder X-ray diffraction pattern of the crystalline solid comprising 7-(4-chlorobutoxy)-3,4-dihydro-(1H)-quinolinone (7-CBQ).

FIG. 2 depicts the infra red spectrum of the crystalline solid comprising 7-CBQ.

FIG. 3 depicts the differential scanning calorimetric (DSC) curve of the crystalline solid comprising 7-CBQ.

FIG. 4 depicts the thermogravimetric analysis (TGA) curve of the crystalline solid comprising 7-CBQ.

### DESCRIPTION OF THE PREFERRED EMBODIMENTS

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

According to the teachings of the present invention, there are provided methods of preparing the crystalline form of 7-CBQ, by crystallizing the crude 7-CBQ, which is obtained essentially as described herein or by any other method known in the art, from different solvents or from solvent mixtures.

Thus, a preferred embodiment of the present invention relates to a method of preparing the crystalline form of 7-(4-chlorobutoxy)-3,4-dihydro-(1H)-quinolinone (7-CBQ), the method comprising:

suspending 7-CBQ in an organic solvent;

heating the suspension to elevated temperature, preferably to reflux;

allowing the thus formed solution to cool gradually;

collecting the obtained crystals by filtration; and

washing the crystals and drying, optionally under reduced pressure.

According to the present invention, the organic solvent used for crystallizing 7-CBQ is selected from the group consisting of methanol, ethanol, 1-propanol, 2-propanol, 1-butanol, 2-butanol, methyl ethyl ketone, diethyl ketone, methyl isobutyl ketone, toluene, methyl acetate, ethyl acetate, isopropyl acetate, butyl acetate, isobutyl acetate, acetonitrile, and mixtures thereof. The preferable solvents for crystallizing 7-CBQ are: methanol, ethanol (absolute or denaturated), 2-propanol, ethyl acetate, toluene, and acetonitrile.

Another preferred embodiment of the present invention relates to a method of preparing the crystalline form of 7-(4-chlorobutoxy)-3,4-dihydro-(1H)-quinolinone (7-CBQ), the method comprising:

suspending 7-CBQ in a first solvent;

heating the suspension to elevated temperature, preferably to reflux;

adding to the thus formed solution a second solvent and allowing the solution to cool gradually;

collecting the obtained crystals by filtration; and

washing the crystals and drying, optionally under reduced pressure.

According to one aspect of the present invention, the first solvent used for crystallizing 7-CBQ is a solvent in which the 7-CBQ is soluble, optionally at elevated temperature, preferably at reflux conditions, selected from the group consisting of methanol, ethanol, 1-propanol, 2-propanol, 1-butanol, 2-butanol, acetone, methyl ethyl ketone, diethyl ketone, methyl isobutyl ketone, toluene, methyl acetate, ethyl acetate, isopropyl acetate, butyl acetate, isobutyl acetate, acetonitrile, and mixtures thereof. Preferably, the first solvent is ethanol or ethyl acetate.

According to another aspect of the present invention, the second solvent used for crystallizing 7-CBQ is a solvent in which the 7-CBQ is not soluble, optionally at reduced temperature, selected from the group consisting of water, hexane, heptane, cyclohexane, and petroleum ether. Preferably, the second solvent is hexane or water.

Another embodiment of the present invention relates to a process for purifying 7-(4-chlorobutoxy)-3,4-dihydro-(1H)-quinolinone (7-CBQ), the process comprising crystallizing the 7-CBQ, which is obtained essentially as described herein or by any other method known in the art, from a solvent or a mixture of solvents for obtaining the purified 7-CBQ, having a purity of at least about 98%, preferably a purity equal to or greater than 99%.

The present invention provides the crystalline form of 7-(4-chlorobutoxy)-3,4-dihydro-(1H)-quinolinone (7-CBQ), and a process for using this crystalline form in the preparation of Aripiprazole thereof.

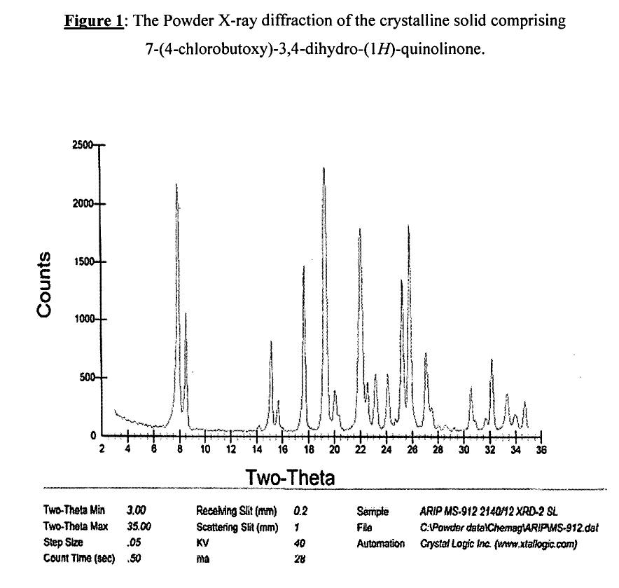

One embodiment of the present invention relates to the crystalline solid comprising 7-(4-chlorobutoxy)-3,4-dihydro-(1H)-quinolinone (7-CBQ), which is characterized by unique powder X-ray diffraction pattern (Table 1, FIG. 1). The strong diffraction peaks at 8.04, 8.61, 15.24, 17.78, 19.44, 22.14, 23.27, 25.33, 25.91, and 27.18±0.2 degrees 2θ are most characteristic of this form.

Table 1 provides the peak positions (2θ deg) and relative intensities (I/I₀) of the powder X-ray diffraction of the crystalline solid comprising 7-(4-chlorobutoxy)-3,4-dihydro-(1H)-quinolinone.

TABLE 1
The peak positions (2θ deg) and relative intensities of the powder
X-ray diffraction of the crystalline solid comprising 7-CBQ.

| Peak Position | Relative |
|---|---|
| (2θ deg) | Intensity (%) |
| 8.04 | 56.1 |
| 8.61 | 26.1 |
| 14.26 | 1.1 |
| 15.24 | 24.4 |
| 15.75 | 7.7 |
| 17.78 | 25.2 |
| 19.44 | 100.0 |
| 20.13 | 7.9 |
| 20.41 | 3.1 |
| 22.14 | 31.6 |
| 22.62 | 9.9 |
| 23.27 | 13.3 |
| 24.20 | 7.4 |
| 24.78 | 1.9 |
| 25.33 | 22.5 |
| 25.91 | 41.3 |
| 27.18 | 15.6 |
| 27.59 | 4.0 |
| 28.70 | 1.3 |
| 30.64 | 8.0 |
| 30.98 | 2.2 |
| 31.75 | 3.4 |
| 32.26 | 9.7 |
| 33.37 | 5.7 |
| 34.06 | 2.5 |
| 34.80 | 6.1 |

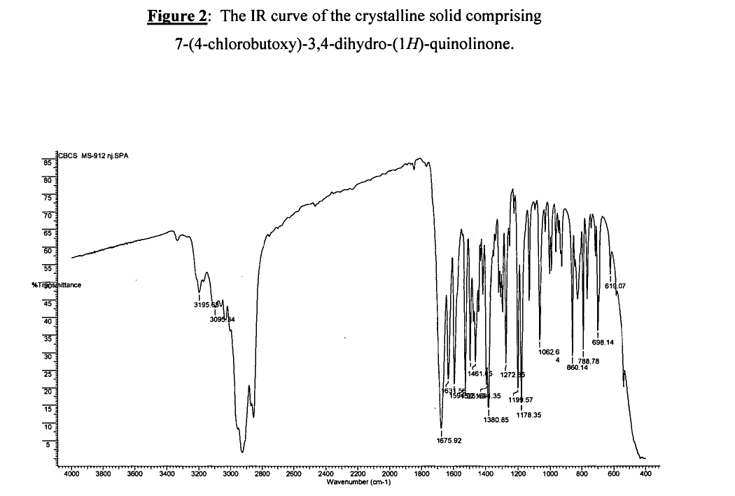

The crystalline solid comprising 7-CBQ is further characterized by having a unique infra-red spectrum with characterizing absorption bands at 3195.63, 3095.34, 1675.92, 1631.56, 1594.92, 1525.49, 1461.85, 1394.95, 1380.85, 1272.85, 1199.57, 1178.35, 1062.64, 860.14, 788.78, 698.14 and 619.07±4 cm⁻¹, as depicted in FIG. 2.

The crystalline solid comprising 7-CBQ is further characterized by having a melting point of 104-105° C.

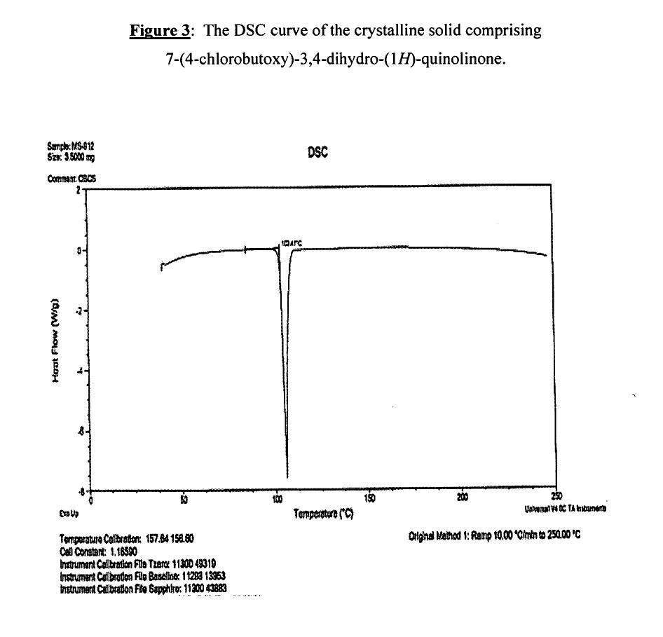

The crystalline solid comprising 7-CBQ is further characterized by having a differential scanning calorimetric (DSC) curve as depicted in FIG. 3. According to this DSC curve, the endothermic peak is at a temperature of 103.41° C., corresponding to the melting of 7-CBQ.

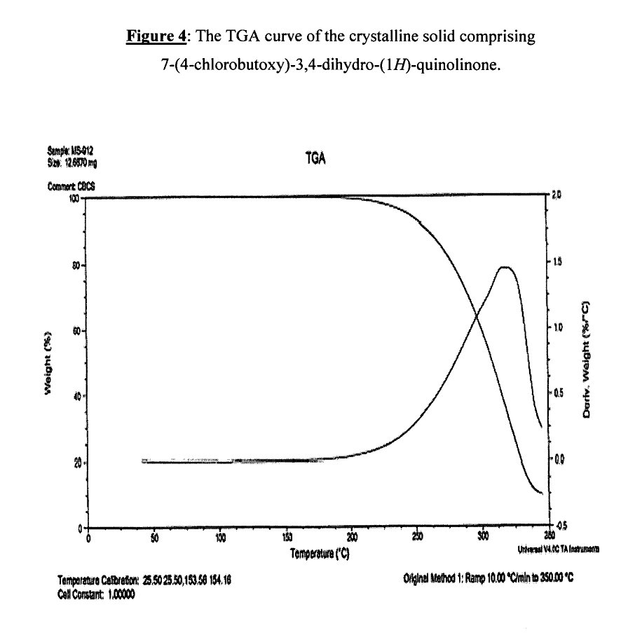

The crystalline solid comprising 7-CBQ is further characterized by having a thermogravimetric analysis (TGA) curve as depicted in FIG. 4.

Although, the following examples illustrate the practice of the present invention in some of its embodiments, the examples should not be construed as limiting the scope of the invention. Other embodiments will be apparent to one skilled in the art from consideration of the specification and examples. It is intended that the specification, including the examples, is considered exemplary only, with the scope and spirit of the invention being indicated by the claims which follow.

### EXAMPLES

Analytical measurements of the 7-CBQ samples were performed using an HPLC system equipped with Phenomenex Luna C8(2) column, 5 μm, 250×4.6 mm, and a UV detector operated on 215 nm. Analyses were performed using the following mobile phase, at flow rate of 1.0 ml/minute, temperature of 30° C., and run time of 15.1 minutes.

Mobile Phase:

Solution A: A buffer solution prepared by adding 85% phosphoric acid to water to obtain a pH of 2.5.

Solution B: acetonitrile

TABLE 2
The gradient program of the 7-CBQ analytical method

| Time, minutes | Buffer % | Acetonitrile % |
|---|---|---|
| 0 | 55 | 45 |
| 11 | 25 | 75 |
| 15 | 25 | 75 |
| 15.1 | 55 | 45 |

The retention time of 7-CBQ is about 8.6 minutes.

The crystalline form of 7-CBQ was characterized by powder X-ray diffraction, which produces a fingerprint of the particular crystalline form. Measurements of 2θ values typically are accurate to within ±0.2 degrees.

X-ray diffraction data was acquired using a PHILIPS X-ray diffractometer model PW1050-70. System description: K1=1.54178Å, voltage 40 kV, current 28 mA, diversion slit=1°, receiving slit=0.2 mm, scattering slit=1° with a Graphite monochromator. Experiment parameters: pattern measured between 2θ=4° and 2θ=30° with 0.05° increments; count time was 0.5 second per increment

The crystalline form of 7-CBQ was further characterized by infra-red spectroscopy run on a Nicolet Avator 360.

The crystalline form of 7-CBQ was further characterized by differential scanning calorimetry (DSC), run on TA Instruments model Q1000, with Universal software version 3.88. Samples were analyzed inside crimped 40 μl aluminum pans. Heating rate for all samples was 10° C./min.

The crystalline form of 7-CBQ was further characterized by thermogravimetric analysis run on TA Instruments model Q500, with universal software version 3.88. Samples were run inside platinum baskets at heating rate of 10° C./min.

### Reference Example 1

### Preparation of 7-(4-chlorobutoxy)-3,4-dihydro-2(1H)-quinolinone (7-CBQ)

A mixture of 7-hydroxy-3,4-dihydro-2(1H)-quinolinone (40 g, 0.245 mole), 1-bromo-4-chlorobutane (85.7 ml, 127.5 g, 0.735 mole, 3 equiv.) and 85% solid potassium hydroxide (21 g, 0.318 mole, 1.3 equiv.) in 2-propanol (200 ml) was heated under reflux for 2 hours. The hot reaction mixture was filtered and the solvent and excess 1-bromo-4-chlorobutane were removed to dryness in vacuum.

2-Propanol (125 ml) was added to the residue thus obtained and the mixture was heated under reflux to obtain a solution. A solution of 47% aqueous sodium hydroxide was added to the hot solution to produce a pH of about 10-11 and the mixture was set aside at 10-15° C. for 6 hours. A colorless precipitate was collected by filtration, washed with the cold mixture of water and 2-propanol (1:3, 50 ml) and water (100 ml) and dried under reduced pressure at 50° C. overnight to obtain crude 7-(4-chlorobutoxy)-3,4-dihydro-2(1H)-quinolinone (56.8 g) in 91.3% yield, having a purity of 98.5% (by HPLC).

### Example 2

### Crystallization of 7-(4-chlorobutoxy)-3,4-dihydro-2(1H)-quinolinone (7-CBQ) from ethanol

In a 100 ml three necked round bottom flask equipped with a reflux condenser, a thermometer and a magnetic stirrer, crude 7-(4-chlorobutoxy)-3,4-dihydro-(1H)-quinolinone (1 gram), obtained as described in reference example 1, was suspended in 20 ml of absolute ethanol. The suspension was heated to reflux to form a solution, maintained at reflux temperature during few minutes and left to cool to room temperature and then to about 5° C. The resulting crystals were filtered, washed with cold ethanol (2 ml) and dried under reduced pressure to obtain 0.9 gram, having the purity of 99.66% (by HPLC).

### Example 3-8

### Crystallization of 7-(4-chlorobutoxy)-3,4-dihydro-2(1H)-quinolinone (7-CBQ) from Different Solvents

Crystallization of crude 7-(4-chlorobutoxy)-3,4-dihydro-(1H)-quinolinone, produced as described in example 1, was carried out using different solvents for obtaining highly pure 7-(4-chlorobutoxy)-3,4-dihydro-(1H)-quinolinone, as shown in table 3.

TABLE 3
Crystallization of 7-(4-chlorobutoxy)-3,4-dihydro-(1H)-quinolinone
using different solvents.

| Example No. | Solvent | Purity (by HPLC) |
|---|---|---|
| 3 | denaturated ethanol | 99.7% |
| 4 | methanol | 99.7% |
| 5 | 2-propanol | 99.65% |
| 6 | ethyl acetate | 99.64% |
| 7 | toluene | 99.65% |
| 8 | acetonitrile | 99.65% |

### Example 9-12

### Crystallization of 7-(4-chlorobutoxy)-3,4-dihydro-2(1H)-quinolinone (7-CBQ) from a Solvent Mixture

In a 100 ml three necked round bottom flask equipped with a reflux condenser, a thermometer and a magnetic stirrer, crude 7-(4-chlorobutoxy)-3,4-dihydro-(1H)-quinolinone (1 gram), obtained as described in example 1, was suspended in 20 ml of a first solvent. The suspension was heated to reflux to form a solution and maintained at reflux temperature during few minutes. Then, heating was ceased and a second solvent was added dropwise (2 ml), and the mixture was left to cool to room temperature and then to about 5° C. The resulting crystals (about 0.8 gram) were filtered, washed with the cold second solvent (2 ml) and dried under reduced pressure.

TABLE 4
Crystallization of 7-(4-chlorobutoxy)-3,4-dihydro-(1H)-quinolinone
using different solvent mixtures.

| Example number | First solvent | Second solvent | Purity |
|---|---|---|---|
| 9 | ethyl acetate | water | 99.75% |
| 10 | ethanol | water | 99.66% |
| 11 | ethyl acetate | hexane | 99.69% |
| 12 | ethanol | hexane | 99.7% |

### Reference Example 13

### Preparation of Aaripiprazole by Reaction of 1-(2,3-dichlorophenyl)piperazine monohydrochloride with 7-(4-chlorobutoxy)-3,4-dihydro-(1H)-quinolinone

A reaction vessel was charged with 7-(4-chlorobutoxy)-3,4-dihydro-(1H)-quinolinone [15.3 g, 0.064 mole], obtained as per example 2, 1-(2,3-dichlorophenyl)piperazine mono hydrochloride (17.8 g, 0.0665 mole), potassium carbonate (9.2 g, 0.0667 mole), tetra-butylammonium bromide (1.8 g), toluene (230 ml) and water (92 ml). The mixture was heated under reflux for 13 hours. Then, the reaction mixture was cooled to about 65° C. and toluene was added (230 ml) and stirring was maintained for 15 minutes. The phases were separated and the aqueous phase was collected (about 96 ml). Water (77 ml) was added to the organic phase and the mixture was stirred at about 65° C. for 15 minutes. The layers were separated and toluene was distilled out (about 184 ml). Ethanol was added (230 ml) in portions at 65° C. to afford a solution. The solution was cooled to about 25° C. and stirred at that temperature for one hour. Then, the solution was cooled to about 5° C. and stirred at that temperature for one hour. The precipitate was collected by filtration and washed with ethanol to obtain a wet solid, which was dried at 60° C. to afford dry crude Aripiprazole (17.6 grams, 65% yield), having a purity of 98%. The crude aripiprazole was crystallized twice from ethanol to obtain the crystallized material having a purity of 99.6%

What is claimed is:

1. A crystalline solid comprising 7-(4-chlorobutoxy)-3,4-dihydro-(1H)-quinolinone (7-CBQ), having a purity of at least about 98%, preferably having a purity equal to or greater than 99.%, and more preferably having a purity equal to or greater than 99.6%.

2. The crystalline solid comprising 7-(4-chlorobutoxy)-3,4-dihydro-(1H)-quinolinone (7-CBQ), according to claim 1, further characterized by unique powder X-ray diffraction pattern, as depicted in table 1 and in FIG. 1, having strong diffraction peaks at 8.04, 8.61, 15.24, 17.78, 19.44, 22.14, 23.27, 25.33, 25.91, and 27.18±0.2 degrees 2θ, which are most characteristic of this form.

3. The crystalline solid comprising 7-CBQ, as defined in claim 2, further characterized by having a unique infra-red spectrum, as depicted in FIG. 2, with characterizing absorption bands at 3195.63, 3095.34, 1675.92, 1394.95, 1631.56, 1594.92, 1525.49, 1461.85, 1380.85, 1272.85, 1199.57, 1178.35, 1062.64, 860.14, 788.78, 698.14 and 619.07±4 cm⁻¹.

4. The crystalline solid comprising 7-CBQ, as defined in claim 2, further characterized by a differential scanning calorimetric curve, as depicted in FIG. 3, having an endothermic peak at about 103.41° C., and a melting point of 104-105° C.

5. The crystalline solid comprising 7-CBQ, as defined in claim 2, further characterized by a thermogravimetric curve as depicted in FIG. 4.

6. A method of preparing a crystalline solid comprising 7-CBQ having a purity of at least about 98%, preferably a purity equal to or greater than 99%, and more preferably a purity equal to or greater than 99.6%, comprising:
suspending 7-CBQ in an organic solvent;
heating the suspension to elevated temperature, preferably to reflux;
allowing the thus formed solution to cool gradually;
collecting the obtained crystals by filtration; and
washing the crystals and drying, optionally under reduced pressure.

7. The method of preparing the crystalline solid comprising 7-CBQ, according to claim 6, wherein the organic solvent used for crystallizing 7-CBQ is selected from the group consisting of methanol, ethanol, 1-propanol, 2-propanol, 1-butanol, 2-butanol, methyl ethyl ketone, diethyl ketone, methyl isobutyl ketone, toluene, methyl acetate, ethyl acetate, isopropyl acetate, butyl acetate, isobutyl acetate, acetonitrile, and mixtures thereof.

8. The method of preparing the crystalline solid comprising 7-CBQ, according to claim 7, wherein the organic solvent used for crystallizing 7-CBQ is selected from the group consisting of methanol, ethanol (absolute or denaturated), 2-propanol, ethyl acetate, toluene, and acetonitrile.

9. A method of preparing a crystalline solid comprising 7-CBQ, having a purity of at least about 98%, preferably a purity equal to or greater than 99%, and more preferably a purity equal to or greater than 99.6%, comprising:
suspending 7-CBQ in a first solvent;
heating the suspension to elevated temperature, preferably to reflux;
adding to the thus formed solution a second solvent and allowing the solution to cool gradually;
collecting the obtained crystals by filtration; and
washing the crystals and drying, optionally under reduced pressure.

10. The method of preparing the crystalline solid comprising 7-CBQ, according to claim 9, wherein the first solvent used for crystallizing 7-CBQ is a solvent in which the 7-CBQ is soluble, optionally at elevated temperature, preferable at reflux conditions, selected from the group consisting of methanol, ethanol, 1-propanol, 2-propanol, 1-butanol, 2-butanol, acetone, methyl ethyl ketone, diethyl ketone, methyl isobutyl ketone, toluene, methyl acetate, ethyl acetate, isopropyl acetate, butyl acetate, isobutyl acetate, acetonitrile, and mixtures thereof.

11. The method of preparing the crystalline solid comprising 7-CBQ, according to claim 10, wherein the first solvent used for crystallizing 7-CBQ is ethanol or ethyl acetate.

12. The method of preparing the crystalline solid comprising 7-CBQ, according to claim 9, wherein the second solvent used for crystallizing 7-CBQ is a solvent in which the 7-CBQ is not soluble, optionally at reduced temperature, selected from the group consisting of water, hexane, heptane, cyclohexane, and petroleum ether.

13. The method of preparing the crystalline solid comprising 7-CBQ, according to claim 12, wherein the second solvent is hexane or water.

14. A process for purifying 7-(4-chlorobutoxy)-3,4-dihydro-(1H)-quinolinone (7-CBQ), the process comprises crystallizing a 7-CBQ, which is obtained essentially as described herein or by any other method known in the art, from a solvent or a mixture of solvents for obtaining the purified 7-CBQ, having a purity of at least about 98%, preferably a purity equal to or greater than 99%, and more preferably a purity equal to or greater than 99.6%.

15. A process for preparing Aripiprazole in high quality and yield by using the crystalline solid comprising 7-CBQ, having a purity of at least about 98%, preferably a purity equal to or greater than 99%, and more preferably a purity equal to or greater than 99.6%.
